(19) United States
(12) Patent Application Publication
(10) Pub. No.: US 2023/0204469 A1
(43) Pub. Date: Jun. 29, 2023

(54) Method and System for Area-Specific Tissue Collection and Analysis

(71) Applicant: The Florida State University Research Foundation, Inc., Tallahassee, FL (US)

(72) Inventors: Sanjay Kumar, Tallahassee, FL (US); Stephen Beesley, Tallahassee, FL (US); Thomas Sullenberger, Tallahassee, FL (US)

(21) Appl. No.: 18/053,949

(22) Filed: Nov. 9, 2022

Related U.S. Application Data
(60) Provisional application No. 63/288,117, filed on Dec. 10, 2021.

(51) Int. Cl. G01N 1/30; G01N 33/68

(52) U.S. Cl. CPC G01N 1/30; G01N 33/6896; G01N 2001/2873

(57) ABSTRACT

An area-specific tissue analysis (ASTA) method and system are disclosed for the precision harvesting and processing of tissue from acute brain slices (referred to herein as “brain chads”), e.g., for the purposes of cell and molecular biology/analysis. The exemplary ASTA system and method can sample tissue from hard-to-reach regions of the brain that has been cut acutely, e.g., into 100 to 500 microns-thick sections, for cell biological analysis.

### RELATED APPLICATION

This application claims priority to, and the benefit of, U.S. Provisional Patent Application No. 63/288,117, filed Dec. 10, 2021, entitled “Method and System for Area Specific Tissue Collection and Analysis,” which is incorporated by reference herein in its entirety.

### BACKGROUND

While there are techniques to transect or dissect small sections of tissue that can be processed for cell biological analysis, e.g., Laser dissection or laser Dissector, such laser-mediated microscopic dissection works only with a monolayer of cells. One technique can collect samples from a single layer of cells that are cultured in a dish to provide a single layer of neurons for analysis.

There are, nevertheless, benefits to being able to collect sections of brain tissues for analysis.

### SUMMARY

An area-specific tissue analysis (ASTA) method and system are disclosed for the precision harvesting and processing of tissue from acute brain slices (referred to herein as “brain chads”), e.g., for the purposes of cell and molecular biology/analysis. The exemplary ASTA system and method can sample tissue from hard-to-reach regions of the brain that has been cut acutely, e.g., into 100 to 500 microns-thick sections, for cell biological analysis. To do so, the exemplary ASTA system and method employ a visually guided core sampling component in combination with a tissue processing component to facilitate precision analysis and tracking of cellular and molecular changes across juxtaposed brain regions (nuclei) and lamina allowing for the detailed characterization of disease-related pathology.

Notably, the exemplary ASTA method and system can be employed to accurately and reproducibly collect tissue samples from specific regions, nuclei, and lamina within the brain as well as other tissue areas described herein for analysis. These brain or tissue chads can be used to extract protein (cytoplasmic and membrane) to run downstream biological assays, e.g., immuno (western) blotting, ELISA, and MEKC. In addition, these brain or tissue chads can be used to extract nucleic acid (DNA or RNA) for use in downstream biological assays, e.g., genotyping, cloning, quantitative polymerase chain reaction (qPCR), and single-cell preparation.

While laser dissection methods, e.g., laser-mediated microscopic dissection, have been employed to collect samples comprising a monolayer of cells, it is inapplicable and/or inadequate 100 to 500 microns-thick sections. The exemplary ASTA method and system can push the boundaries of cell biological analysis in providing brain chads as well as tissue samples that retain a physiologically and morphologically complete representation of the complex brain tissue and other tissue samples in these 100 to 500 microns-thick sections. Indeed, in 100 to 500 microns-thick sections, the collected brain chads can include multiple layers of cells that still retain their organization as a neural circuit in which cell biology and electrophysiology can be studied. The brain chads can include neurotransmitters and neuromodulators in levels representative of the brain region.

A biological sample may be obtained using the exemplary ASTA method and/or system from a eukaryotic organism, for example, a mammal, including humans, cows, pigs, chickens, turkeys, ducks, geese, dogs, goats, and the like. Any tissue appropriate for use in accordance with the invention may be used, for instance, skin, brain, spinal cord, adrenals, pectoral muscle, lung, heart, liver, crop, duodenum, small intestine, large intestine, kidney, spleen, pancreas, adrenal gland, bone marrow, lumbosacral spinal cord, or blood.

The collected tissue may be used in the study or treatment of neurological diseases, e.g., associated with immune failure related to increasing levels of disease-causing factors that exceed the ability of the immune system to contain or a situation in which immune function deteriorates or is suppressed concomitantly with disease progression, due to factors indirectly or directly related to the disease-causing entity.

MDSCs can cause T-cell deficiency by suppressing effector T-cell activity, thus promoting neurodegenerative disease associated with immune failure.

Representative examples of Protein Aggregation Disorders or Proteopathies include Protein Conformational Disorders, Alpha-Synucleinopathies, Polyglutamine Diseases, Serpinopathies, Tauopathies or other related disorders. Other examples of neurological diseases or include, but are not limited to, Amyotrophic Lateral Sclerosis (ALS), Huntington's Disease (HD), Parkinson's Disease (PD), Spinal Muscular Atrophy (SMA), Alzheimer's Disease (AD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCAT), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, subacute sclerosing panencephalitis, cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias including tremor (such as rest tremor, postural tremor and intention tremor), micro-concussion related injuries (e.g., from mild brain trauma exhibited from sports injury), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as iodiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; obesity, bulimia nervosa and compulsive eating disorders; pain including bone and joint pain (osteoarthritis), repetitive motion pain, dental pain, cancer pain, myofacial pain (muscular injury, fibromyalgia), perioperative pain (general surgery, gynecological), chronic pain, neuropathic pain, posttraumatic pain, trigeminal neuralgia, migraine and migraine headache; obesity or eating disorders associated with excessive food intake and complications associated therewith; attention-deficit/hyperactivity disorder; conduct disorder; mood disorders including depressive disorders, bipolar disorders, mood disorders due to a general medical condition, and substance-induced mood disorders; muscular spasms and disorders associated with muscular spasticity or weakness including tremors; urinary incontinence; amyotrophic lateral sclerosis; neuronal damage including ocular damage, retinopathy or macular degeneration of the eye, hearing loss or tinnitus; emesis, brain edema and sleep disorders including narcolepsy, and apoptosis of motor neuron cells. Illustrative examples of the neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. The neuropathic pain includes pain caused by either central or peripheral nerve damage. And it includes the pain caused by either mononeuropathy or polyneuropathy.

### BRIEF DESCRIPTION OF THE DRAWINGS

FIG. 1 illustrates an example area-specific tissue analysis (ASTA) method and flowchart, according to one implementation.

FIG. 2 shows images of brain slices, reproduced from the method of FIG. 1, according to one implementation.

FIG. 3 shows a coring operation according to one implementation.

FIG. 4 shows a manual tissue corer/harvesting instrument and area-specific tissue analysis (ASTA) rig, according to one implementation.

FIG. 5 shows a motorized tissue corer/harvesting instrument, according to one implementation.

FIG. 6 shows an automated motorized tissue corer/harvesting instrument, according to one implementation.

FIG. 7 shows experimental results for the use of an area-specific tissue analysis (ASTA) to assay the distribution of various subunits of the NMDA receptor in hippocampus (CA1 region), according to one implementation.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entirety and to the same extent as if each reference was individually incorporated by reference.

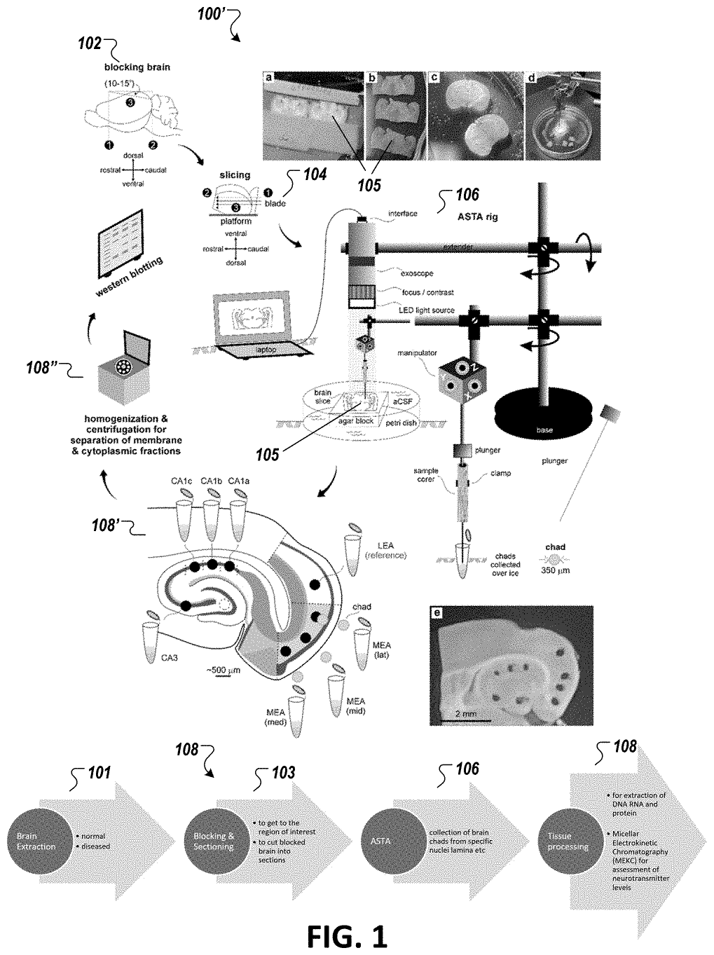

FIG. 1 shows an example area-specific tissue analysis (ASTA) method 100 (shown as 100′) to harvest and process tissue, e.g., from acute brain slices (“brain chads”), e.g., for the purposes of cell and molecular biology/analysis. FIG. 1 shows a flow chart 108 of the example area-specific tissue analysis (ASTA) method to harvest and process tissue from acute brain slices (“brain chads”), e.g., for the purposes of cell and molecular biology/analysis. The method 100′ described in the context of brain tissue, first, includes extracting 101 the brain from an animal model or a subject. The brain and associated tissue can be normal/healthy, or it can be diseased.

In the example shown in FIG. 1, the method 100′ then involves cutting 102 and/or sectioning 104 (also referred to herein as 103 and shown as “blocking” 102 and “slicing” 104) the brain into slices or blocks 105 having the region of interest. In some embodiments, the brain is sectioned or sliced (102, 104) into 100 to 500 microns-thick sections, having the specific regions, nuclei, and lamina within the brain of interest. In some embodiments, the brain is sectioned or sliced in sections 105 which are about 100 microns thick, about 120 microns thick, about 140 microns thick, about 160 microns thick, about 180 microns thick, about 200 microns thick, about 220 microns thick, about 240 microns thick, about 260 microns thick, about 280 microns thick, about 300 microns thick, about 320 microns thick, about 330 microns thick, about 340 microns thick, about 360 microns thick, about 380 microns thick, about 400 microns thick, about 420 microns thick, about 440 microns thick, about 460 microns thick, about 480 microns thick, about 500 microns thick. In some embodiments, the brain can be sectioned or sliced into sections greater than 500 microns thick, e.g., about 600 microns thick, about 700 microns thick, about 800 microns thick, about 900 microns thick, or 1000 microns thick. In some embodiments, the brain is excised in an ice-cold cutting solution (not shown).

An example sectioning tool that may be used in the exemplary method is the microslicer (VTI000S, Leica, Germany). The sliced tissue is kept alive and equilibrated in oxygenated artificial cerebrospinal fluid (aCSF) (e.g., containing (in mM) 126 NaCl, 26 NaHCO3, 3 KCl, 1.25 NaH2PO4, 2 MgSO4, 2 CaCl2, 0.25 L-Glutamine, and 10 D-glucose (pH 7.4)) prior to being subjected to the harvesting operation. In some embodiments, the excised tissue can be stored in a specimen preservation chamber, e.g., as described in U.S. Pat. No. 8,722,403.

The method 100′ then includes performing 106, 108 an area-specific tissue analysis (ASTA) (shown via 108′ and 108″) and harvesting operation (106) to collect or harvest brain chads from the sliced sections comprising the specific regions, nuclei, and lamina within the brain of interest.

Tissue Collection:

The area-specific tissue analysis (ASTA) method and system are described in relation to an animal model, though it can be readily applied to human tissues. In the example shown in FIG. 1, a rat animal model is deeply anesthetized with urethane (1.5 g/kg ip) and decapitated. Horizontal slices (˜600 μm thick) are cut from the excised brain (in the example, excised by blocking) in ice-cold cutting solution (in mM), comprising: 230 sucrose, 10 D-glucose, 26 NaHCO₃, 2.5 KCl, 1.25 NaH2PO4, 10 MgSO4, and 0.5 CaCl2 (equilibrated with 95% 02-5% CO2). Cerebellar samples are collected for use as a control in this example. The cut slice (e.g., 105) (˜6-8 slices of MEA-containing tissue per brain) is carefully placed on a 3% agar block made with 0.9% artificial cerebral spinal fluid and, using a DinoLight microscope for guidance, and holes are punched using a sample corer (Fine Science Tools; 350 nm diameter) in layer 3 MEA: medial MEA—close to the parasubiculum, middle MEA and lateral MEA—close to the LEA. Holes can be punched in the adjacent LEA, CA1a, CA1c and CA3 of the hippocampus or anywhere desired in the brain slices.

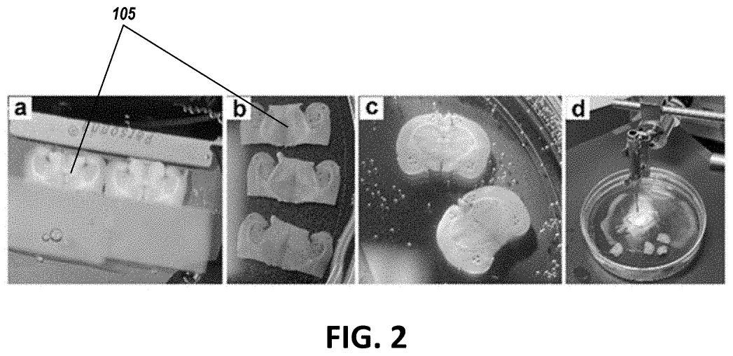

FIG. 2 (reproduced from FIG. 1) shows an example of brain slices (e.g., 105) that are excised from an animal model (panel A). The sliced samples are immediately placed in aCSF (panel B) to keep them alive and oxygenated. The sliced samples (e.g., 105) are then moved onto a cutting substrate comprising a gelling agent substrate (e.g., agar+aCSF mixture) and in a cutting solution (e.g., aCSF) (panel C). The coring operation is performed in an open environment on the sliced samples while the sample is seated on the cutting substrate and bathed in the cutting solution.

Example Method of Operation

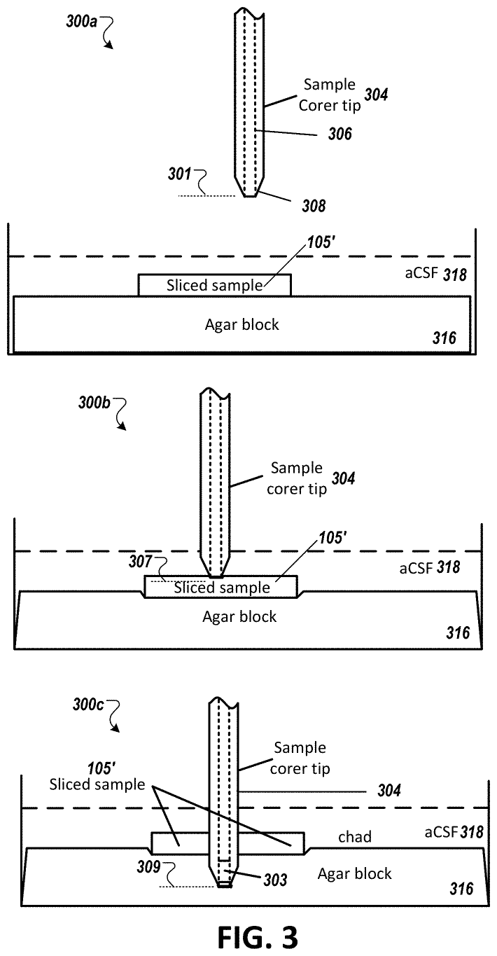

FIG. 3 shows an example of a coring operation that can be performed. In the first diagram 300a of FIG. 3, the sample corer 304 is located at a first position 301 above the sliced sample 305. The sample corer 304 (shown as “Sample Corer tip” 304) comprises an elongated hollow probe 306 with a taper opening 308. An example is the sample corer (Fine Science Tools; 350 nm diameter). Other sample corer sizes and configurations may be used. The sliced sample 105 (shown as 105′) is placed on a cutting substrate 316, e.g., comprising a gelling agent substrate (shown as “Agar block” 316) and in a cutting solution 318 (shown as artificial cerebrospinal fluid “aCSF” 318).

In the second diagram 300b, the sample corer 304 is moved from the first position 301 to a second position 307 via a guidance system, for example, a three-axis manipulator (not shown) capable of moving the sample corer 304 in three dimensions. In the second position 307, the sample corer 304 has entered into the cutting solution 318 and has touched the sliced sample 105′.

In the third diagram 300c, the sample corer 304 is moved from the second position 307 to a third position 309 by the guidance system. In the third position 309, the sample corer 304 has cut through the sliced sample 105′ and into the cutting substrate 316. The sample corer 304, therefore, has removed a chad 303 from the sliced sample 305.

Tissue Corer/Harvesting System

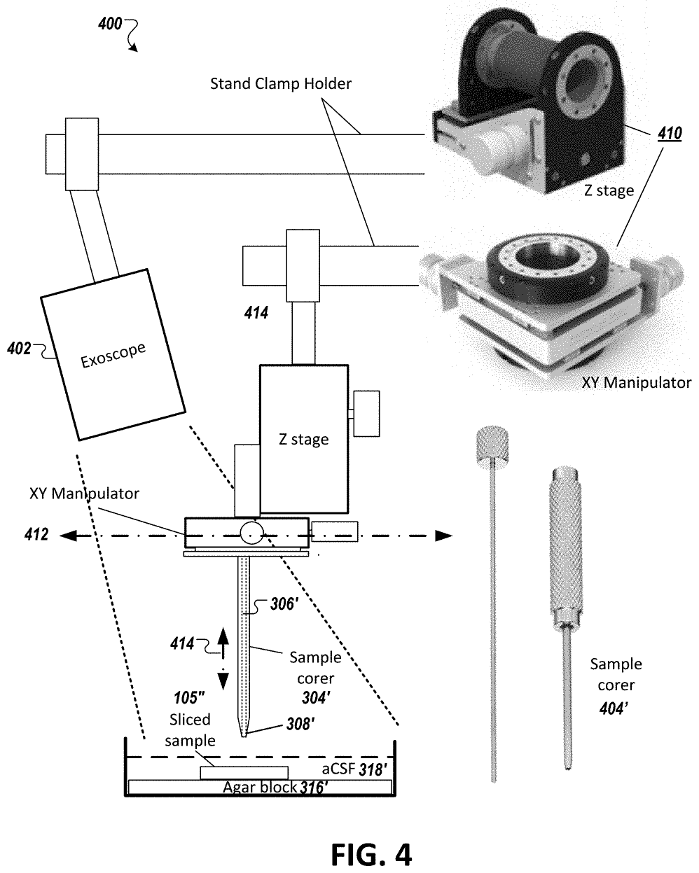

FIG. 4 shows an example of the tissue corer/harvesting instrument 400, e.g., corresponding to “ASTA rig” of FIG. 1, that is hand manipulated. The instrument 400 includes an exoscope 402, or other imaging system, configured to visually guide the harvesting of sample chads 303 from a sliced sample 105. The instrument 400 further includes a sample corer 304 (shown as 304′) comprising the elongated hollow probe 306 (shown as 306′) with a taper opening 308 (shown as 308′). An example is the sample corer (Fine Science Tools; 350 nm diameter). Other sample corer sizes and configurations may be used.

In the example shown in FIG. 4, I instrument 400 includes a three-axis manipulator 410 fixably coupled to the sample corer 304′, the three-axis manipulator 410 being configured to move along (i) a first plane 412 parallel to a sliced sample 405, e.g., of 100-micron to 500-micron thickness, and (ii) a second plane 414 that bisects the sliced sample 105 (shown as 105″) where the sliced sample 105″ is placed on a cutting substrate 316, e.g., comprising a gelling agent substrate (shown as “Agar block” 316′) and in a cutting solution 318 (shown as artificial cerebrospinal fluid “aCSF” 318′).

The exoscope 402, in some embodiments, is a digital imaging system that includes optical, sensor, and electronic components that can produce magnified, three-dimensional images, e.g., of an open surgical field, particularly microsurgeries. The exoscope 402 may include an interface for a monitor to display the acquired images/video and/or user interface to receive commands for its operations, e.g., pan, zoom, and/or change configurations.

In the example shown in FIG. 4, the three-axis manipulator 410 includes an XY manipulator capable of translating the sample corer 404 along a first plane 412 parallel to a sliced sample 405. The three-axis manipulator 410 further includes a Z Stage capable of translating the sample corer 304′ along a second plane 414 that bisects the sliced sample 105″. In some embodiments, the XY manipulator and/or the Z Stage may be operated by a user in a handheld manner. In some embodiments, the manipulator may only move in a single dimension (e.g., the x-direction) or only in two directions (e.g., the x-direction and the z-direction).

Motorized Tissue Corer/Harvesting System

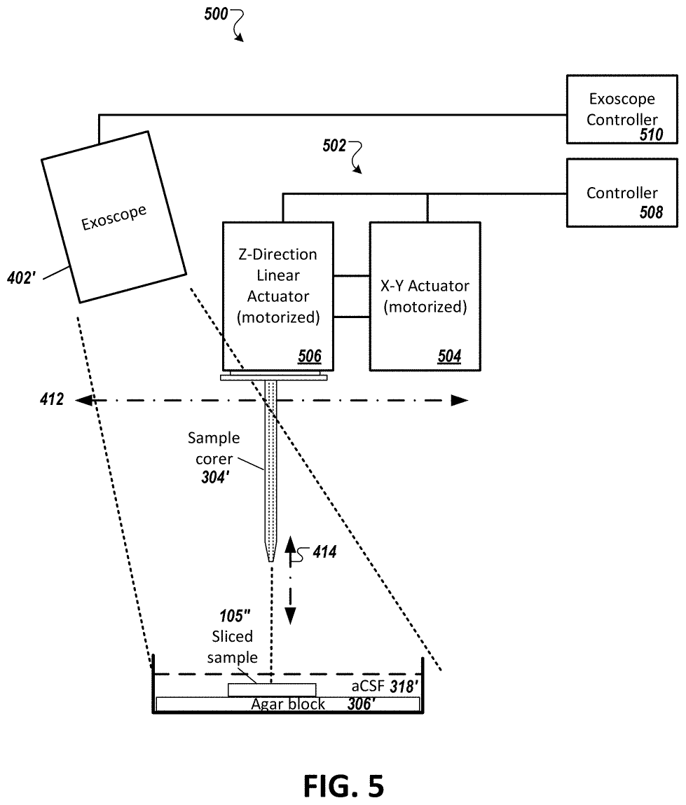

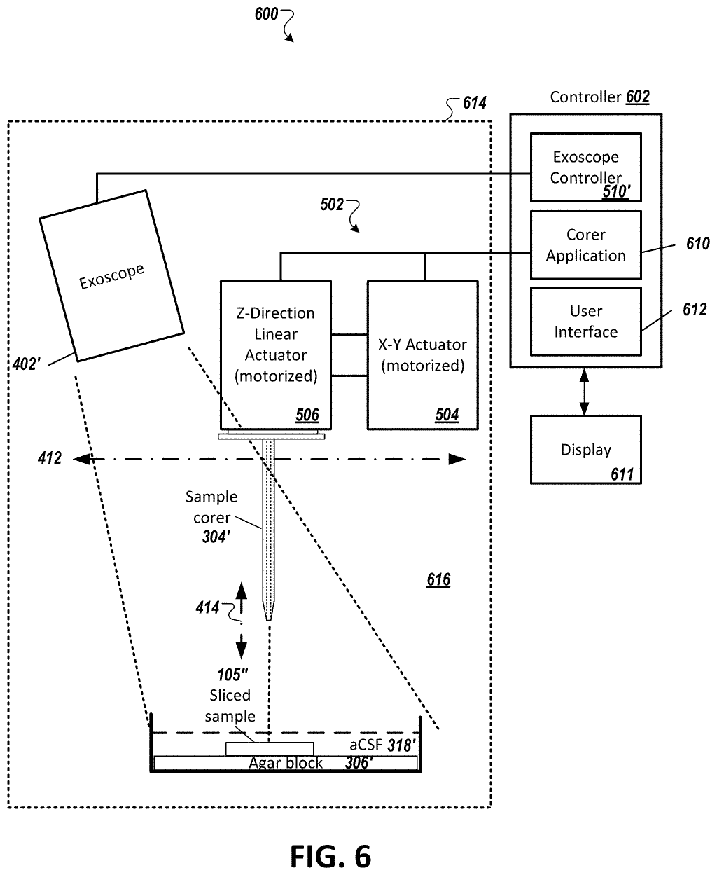

In FIG. 4, the actuator is manually actuated. FIGS. 5 and 6 each shows an embodiment of the instrument in which the actuator is motorized. In the example shown in FIG. 5, the tissue corer/harvesting instrument 400 (shown as 500) includes the exoscope 402 configured to visually guide the harvesting of sample chads (e.g., 303) from a sliced sample 105 (shown as 105″). Instrument 500 also includes the sample corer 304 (shown as 304′).

Instrument 500 describes an embodiment in which a user or technician can operate a motorized system to excise sample cores for ASTA analysis. Instrument 500 includes motorized manipulator 502, which can adjust the location of sample corer 504 via the controller 508. Instrument 500 also includes exoscope 402 (shown as 402′), which can be adjusted by the exoscope controller 606. The user can control the operation of instrument 500 by inputting controls into controller 508 and exoscope controller 510. In some implementations, the controllers 508, 510 may be integrated into a single interface and/or controller. The exoscope controller 510 can send signals to the exoscope 402′, e.g., to adjust the optical or digital zoom, pan, image resolution, optical and/or digital filters, and other data related to the image captured and produced by the exoscope 402′. The instrument 500 may include mechanical adjustments that can operate with the motorized system.

The instrument 500 includes a motorized manipulator 502 that is coupled, fixably or releasably, to the sample corer 304′. In some embodiments, the sample corer 304′ can be coupled and decoupled from the motorized manipulator 502 via a connection assembly that can quickly couple and decouple the sample corer 304′.

The motorized manipulator 502 is operatively coupled to the controller 508 and is configured to move along a first plane 412 parallel to a sliced sample 105″. The motorized manipulator 502 may include a second actuator 506 (shown as “Z-Direction Linear Actuator” 506) that can move in the second plane 414 that bisects the sliced sample 105″. In various implementations, the instrument 500 may employ motorized components for movement in all three directions, or only two directions (e.g., only the x-direction and y-direction, or only the x-direction and z-direction), or only 1 direction (e.g., only in the z-direction), depending on the particular use case.

The instrument 500 includes a controller 508 configured to communicate with the motorized manipulator 502 such that an input to controller 508 can drive a portion of motorized manipulator 502 to move the sample corer 304′. For example, an input to controller 508 may activate a motor within the XY Actuator 504, causing the sample corer 304′ to translate in an x-direction or a motor in the Z-direction Actuator 506 to move the sample corer 304′ to move in the z-direction 414.

Automated Tissue Corer/Harvesting System

FIG. 6 shows another configuration of an automated motorized instrument 600. The instrument 600 includes the motorized manipulator 502, e.g., comprising actuators 504, 506, that can operate with a controller 602.

The controller 602 includes a software application 610 (shown as “Corer Application” 610) that includes the controls for the motorized manipulator 502. In the example shown in FIG. 6, the software application 610 interfaces to the exoscope controller 510 (shown as 510′). In this example, the exoscope controller 510′ may include a set of drivers for the exoscope 402′. In other embodiments, the exoscope controller 510′ is a standalone software application for controlling the exoscope 402′, which has APIs (application program interfaces) for executing commands to the software. The controller 602 may output, via displays 611, the user interface 612 for control of the motorized manipulator 502.

The controller 602 and corer application 610, in some embodiments, are configured to present images/video acquired of the sliced sample 105″ (as located on the cutting substrate 306′ and cutting solution 318′. The corer application 610 can provide the user with commands to select a region of the sliced ample 105″. The corer application 610 can include instructions to generate a trajectory, via actuation, of the motorized manipulator 502 for movement of the sample corer 304′ in the selected region. In some embodiments, the instrument 600 can then direct sampling of the chads (e.g., 303) via the motion of the corer in the z-direction 414. In other embodiments, the instrument 600 can prompt the user to initiate the sampling of the chads (e.g., 303) via the motion of the corer in the z-direction 414.

In some embodiments, the instrument 600 includes a housing 614 that can isolate the ASTA operation from the external environment. In some embodiments, housing 614 includes a temperature regulation unit configured to cool the environment 616 of the instrument 600. The temperature regulation unit may be controlled via the controller 602.

### Example #1: Protein Extraction

As described above, the area-specific tissue analysis (ASTA) method and system, as described herein, can be applied to animal models and human tissues.

The process can be used for protein extraction. A collection of brain chads may be study to determine the levels of excitatory, and inhibitory neurotransmitter, which is basically what is disrupted, e.g., in epilepsy, where the excitation goes up and inhibition goes down. More sophisticated analysis, e.g., sampling of neuro modulators, neurotransmitters, and also more importantly, proteins, can also be performed since the synapses and receptors are preserved in the collected brain chads, which are proteinaceous entities. Indeed, the exemplary system and method can facilitate the sampling of receptors are that up-regulated, and what receptors are down located. This sampling from control animals and from animals, for example, that have disease, for example, temporal lobe epilepsy, but it could be Alzheimer's, it could be Parkinson's, it could be PTSD, or what have you. Other analysis as described herein can be performed.

In the example shown in FIG. 1, a rat animal model is deeply anesthetized with urethane (1.5 g/kg ip) and decapitated. Horizontal slices (˜600 μm thick) are cut from the excised brain (in the example excised by blocking) in ice-cold cutting solution (in mM), comprising: 230 sucrose, 10 D-glucose, 26 NaHCO₃, 2.5 KCl, 1.25 NaH2PO4, 10 MgSO4, and 0.5 CaCl2 (equilibrated with 95% 02-5% CO2). Cerebellar samples are collected for use as a control in this example. The cut slice (e.g., 105) (˜6-8 slices of MEA-containing tissue per brain) is carefully placed on a 3% agar block made with 0.9% artificial cerebral spinal fluid and, using a DinoLight microscope for guidance, holes are punched using a sample corer (Fine Science Tools; 350 nm diameter) in layer 3 MEA: medial MEA—close to the parasubiculum, middle MEA and lateral MEA—close to the LEA. Holes can be punched in the adjacent LEA, CA1a, CA1c and CA3 of the hippocampus or anywhere desired in the brain slices.

To further maintain the tissue during the protein extraction, the tissue punches (e.g., chads) can be made and kept in ice-cold cutting solution (above) until all punches are collected. The remaining steps can thus be all conducted on ice, e.g., or at 4° C.

While an estimation of tissue mass can be difficult due to the small quantity collected, it is noted that approximately 16×350 μm (diameter) tissue punches (e.g., chads) can provide sufficient material for protein or RNA/DNA extraction. Once all punches are collected, the cutting solution can be removed, and tissue can be washed once with wash buffer (Mem-PER™ Plus Membrane Protein Extraction Kit; ThermoFisher Scientific) and extraction of both cytoplasmic and membrane fractions are as follows using the Mem-PER™ Plus Membrane Protein Extraction Kit. The wash buffer can be removed and permeabilization buffer (e.g., 30 μL) with added protease inhibitors (Sigma) can be added and the tissue can be homogenized using a motorized pestle homogenizer (VWR), using approximately 10 strokes up and down. For extracting RNA or DNA nuclease-free reagents and consumables can be employed. The samples can be briefly centrifuged and allowed to rotate, e.g., for 10 minutes at 4° C., to fully lyse the cells. Following this, the samples can be centrifuged, e.g., for 15 minutes at 16,000 g at a processing temperature that can preserve the brain tissue, e.g., at 4° C. The resulting supernatant (cytoplasmic fraction) can be removed and mixed, e.g., with 10 ml 4×SDS sample buffer (in mM): 200 Tris (pH 6.8), 40% glycerol, 10 EDTA (pH 8), 8% SDS, 10% β-mercaptoethanol, 8 mg bromophenol blue and boiled for 3 minutes at 100° C. Prior to mixing with the sample buffer, a small amount of supernatant can be taken for protein quantification e.g., bicinchoninic acid (BCA) assay. The tissue pellet can then be re-homogenized, using the motorized pestle, in 30 μl membrane buffer with added protease inhibitors, using approximately 5 strokes up and down. Again, the samples can be briefly centrifuged and then allowed to rotate for 30 minutes at 4° C. to release proteins from the membrane. On completion, the samples can be centrifuged for 15 minutes at 16,000 g at 4° C. The resulting supernatant (membrane fraction) can be removed and mixed with 10 ml 4×SDS sample buffer and boiled for 3 minutes at 100° C. As with the cytoplasmic fraction, a small volume of supernatant can be removed prior to the addition of sample buffer for protein quantification. All samples can be stored, e.g., at −80° C. until use.

### Example #2: BCA Assay

BCA solution (VWR) can be added to a well plate, e.g., 96-well plate. In some embodiments, 0.5, 1, 1.5, 2 μg bovine serum albumin may be added to the BCA solution (in duplicate) to form a standard curve. For the unknown samples, 2 μl of sample are added to the BCA solution. The plate may be briefly mixed by shaking and left at room temperature, e.g., for 20-30 minutes prior to reading at 595 nm.

### Example #3: Immuno (Western) Blotting

Polyacrylamide gels must be made with respect to the size of the protein of interest. To detect NMDA receptor subunits, a 6% polyacrylamide gel can be casted. The membrane protein fraction may be added to the gel and allowed to migrate in running buffer (mM: 25 Tris base, 191 glycine, 0.1% SDS) at 180V for approximately 75 minutes, or until the dye front falls off the gel. The gel may then be transferred to polyvinylidene difluoride (PVDF) membrane using transfer buffer (in mM): 25 Tris, 191 glycine at 75V for 90 minutes at 4° C. The membrane may be blocked for 30 minutes in 5% fat-free milk dissolved in tris buffered saline (pH7.4-7.6) with 0.05% tween-20 (TBST; mM: 500 Tris base, 1500 NaCl) and the primary antibody (diluted with TBST, 0.5% milk and 0.02% sodium azide as preservative) may be added and left to incubate overnight at room temperature with gentle agitation. Primary antibodies (Millipore, unless state otherwise) can be used, e.g., at a 1 in 1000 dilution: anti-NMDAR1 (rabbit, clone 1.17.2.6); anti-NR2B (mouse, clone BWJHL); anti-NR2A (rabbit, clone A12W); and anti-NR3A (rabbit) and Na+/K+ ATPase (rabbit, Lot 5, Cell Signaling Technologies). The following day the primary antibody may be removed, and membrane washed 3×5 minutes. Secondary antibody, goat anti-rabbit IRDye 800CW or donkey anti-mouse IRDye 680LT (LI-COR) is then added and allowed to incubate in the dark for 1 hour with gentle agitation. Membranes are again washed 3×5 minutes in TBST and then imaged on the LiCor Odyssey CFX imager.

### Example #4: RNA Extraction

The primary use for RNA extraction is for quantitative polymerase chain reaction (qPCR). Tissue punches may be collected and washed once in wash buffer (Mem-PER™ Plus Membrane Protein Extraction Kit; ThermoFisher Scientific) and stored at −80° C. until use. To extract RNA, TRIzol™ Reagent (Invitrogen) may be used and according to the manufacturers' instructions. DNA and protein may be extracted using TRIzol™ Reagent but have not been tried with the punches. All steps may be performed at room temperature (RT), but centrifugation is done at 4° C. The working area may be cleaned and made as RNase-free as possible; working in a controlled environment, e.g., a laminar flow hood may be optimal. All pipettes, tip and tubes should be RNase-free. TRIzol™ Reagent (1 ml) may be added to the tissue and homogenized using a motorized pestle (nuclease-free) homogenizer (VWR). The homogenate may be allowed to incubate, e.g., for 5 minutes at RT, and 0.2 ml chloroform is added and the tube is mixed and incubated for 2-3 minutes before centrifugation at 12,000 g for 15 minutes at 4° C. After separation, the upper aqueous phase may contain RNA (interphase and organic phase contains DNA and proteins) and may need to be transferred to a new RNase-free tube. As the concentration of RNA is small, a carrier can be used to better precipitate the RNA. Here glycogen may be employed in which 5 μg may be added to the transferred aqueous phase. Following this, 0.5 ml (per 1 ml of TRIzol™ Reagent) of isopropanol may be added to the aqueous phase and incubated at RT for 10 minutes. The solution may then be centrifuged at 12,000 g for 10 minutes at 4° C. to precipitate the RNA:glycogen which forms as a white pellet. The pellet is washed in 1 ml 75% ethanol (per 1 ml TRIzol™ Reagent) and centrifuged at 7,500 g for 5 min at 4° C. This ethanol wash may be repeated twice or more (total of 3 washes) to remove as much guanidinium thiocyanate as possible. Once the ethanol is removed the pellet may be air dried for 5-10 mins while being careful not to over dry. The pellet may be resuspended in 15 μl of RNase-free water and placed at 55° C. for 10 minutes. The RNA in the resulting solution may be quantified using a Nanodrop.

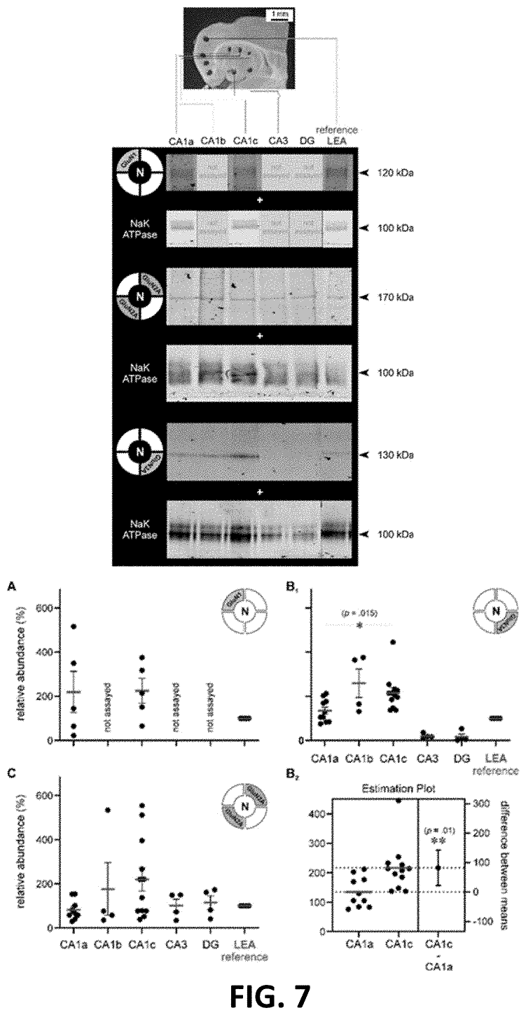

Feasibility: Example of the use of ASTA to assay the distribution of various subunits of the NMDA receptor in hippocampus (CA1 region) (FIG. 7).

Discussion

The exemplary system and method can be used to facilitate new areas of cell biology research and study. The exemplary system and method can be used for the study of neurological disease among other pathologies. An animal model can be made epileptic, e.g., by invoking recurrent seizures. Seizure activity can be recorded, and then animal brain can be excised for analysis. Because the temporal lobe, and other regions of the brain, is made up of several divisions, the instant harvesting method can be used to collect in specific regions of the brain, e.g., in the enteral contacts and hippocampus regions of the brain, to which pathology that gives rise to these seizures or pathologies are manifested.

The exemplary system and method is beneficial in providing samples from brain areas that can be very difficult to access and facilitate the collection of samples that maintain the electrophysiology properties and physiological of the brain slices. Notably, the collected brain chads can be used to observe the cells and circuits that are involved with the pathology either electrophysiologically or by staining the tissue, e.g., with antibodies and with agents that facilitate the visualization of the cells and circuits, e.g., that are involved in temporal lobe epileptogenesis. The exemplary system and method can be used to take small sections of tissues from specific regions within the brain slices to produce samples that can be analyzed, e.g., by isolating DNA, isolating mRNA, isolating neurotransmitter and neuro modulators and their levels in the specific regions.

While there are current methods and systems to transect or dissect small sections of tissue that can be processed for cell biological analysis, e.g., Laser dissection or laser Dissector, such laser mediated microscopic dissection works only with a mono layer of cells. For example, such system can be used to collect samples from a layer of cells that are cultured in a dish to provide a single layer of neurons. However, these systems facilitate the dissection of micro regions as small brain chads from very specific locations within the region of interest.

Visualization of these hard-to-reach areas is an important feature. Because the exemplary method involves micro dissection of small regions of the brain, the technician has to be able to view the tissue under a microscope. But when a slice of tissue is placed under a microscope, there is a technical challenge associated with the working distance to the sample. The use of an exoscope provides a working distance for such tissue harvesting (e.g., 8 to 10 inches as an example).

The collection of chads using a core sampler is also an important feature. Sample corer is akin to what a physician might use to make a biopsy of a sample. The three-axis manipulator facilitates very fine motor and location control of the core sampler to then punch out a brain chad sample. And then, under visual guidance, the chads can be moved to a solution that can be used, e.g., homogenization, and getting the brain sample ready for further cell biological processing.

A collection of brain chads may be study to determine the levels of excitatory, and inhibitory neurotransmitter, which is basically what is disrupted, e.g., in epilepsy, where the excitation goes up and inhibition goes down. More sophisticated analysis, e.g., sampling of neuro modulators, neurotransmitters, and also more importantly, proteins, can also be performed since the synapses and receptors are preserved in the collected brain chads, which are proteinaceous entities. Indeed, the exemplary system and method can facilitate the sampling of receptors are that up-regulated, and what receptors are down located. This sampling from control animals and from animals, for example, that have disease, for example, temporal lobe epilepsy, but it could be Alzheimer's, it could be Parkinson's, it could be PTSD, or what have you. The exemplary area specific tissue analysis is very enabling in the sense that it allows visualization of the region of interest and sampling very specifically. This solves the problem of specificity, meaning that a researcher can go collect, e.g., six layers of the cortex, to which any layer can be sampled, say, at layer three, and then to sample out that layer, for analysis within the laminate or cross laminate. This technique thus can provide very precise information of what the pathology is, and where it is manifest and how its manifest.

In some embodiments, the exoscope is configured with LEDs for lighting. In other embodiments, the exemplary system and method may incorporate differential interference contrast (DIC) that employs polarize light.

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The following patents, applications and publications as listed below and throughout this document are hereby incorporated by reference in their entirety herein.

What is claimed is:

1. A method to collect brain chads consisting of multiple layers of cells, the method comprising:
extracting a brain region from a subject;
slicing a 100-micron to 500-micron thick section from the brain region;
placing the sliced 100-micron to 500-micron thick section on a cutting substrate comprising a gelling agent substrate and in a cutting solution; and
coring the sliced 100-micron to 500-micron thick section to form a brain chad;
wherein the brain chad is employed in subsequent cell and molecular biology/analysis.

2. The method of claim 1, wherein the sliced 100-micron to 500-micron thick section is cored by a mechanically-guided actuator.

3. The method of claim 2, wherein the actuator is motorized.

4. The method of claim 2, wherein the actuator is manually actuated.

5. The method of claim 1, wherein the sliced 100-micron to 500-micron thick section is cored under a visual guide having a wide-field view of the sliced 100-micron to 500-micron thick section.

6. The method of claim 1, wherein the sliced 100-micron to 500-micron thick section is immersed in the cutting solution comprising artificial cerebrospinal fluid (aCSF) when placed on the cutting substrate.

7. The method of claim 1, wherein the gelling agent substrate comprises predominantly of agar mixed with artificial cerebrospinal fluid (aCSF).

8. The method of claim 1, wherein the subsequent cell and molecular biology/analysis includes precision analysis and tracking of cellular and molecular changes across juxtaposed brain regions (nuclei) and lamina allowing for the detailed characterization of disease-related pathology.

9. The method of claim 1 further comprising:
performing protein extraction on the brain chad.

10. The method of claim 1 further comprising:
performing immuno (Western) blotting on the brain chad.

11. The method of claim 1 further comprising:
performing BCA assay on the brain chad.

12. The method of claim 1 further comprising:
storing the extracted brain region alive and equilibrated in oxygenated artificial cerebrospinal fluid (aCSF) prior to being subjected to the harvesting operation.

13. The method of claim 1, wherein the sliced 100-micron to 500-micron thick section is cored under differential interference contrast (DIC).

14. The method of claim 1, wherein the subsequent cell and molecular biology/analysis includes precision analysis and tracking of cellular and molecular changes across juxtaposed brain regions (nuclei) and lamina allowing for the detailed characterization of Parkinson's Disease (PD), Spinal Muscular Atrophy (SMA), Alzheimer's Disease (AD), or Epilepsy.

15. The method of claim 1, wherein the subsequent cell and molecular biology/analysis includes precision analysis and tracking of cellular and molecular changes across juxtaposed brain regions (nuclei) and lamina allowing for the detailed characterization of neurological diseases.

16. A tissue collection system comprising:
an exoscope configured to visually guide harvesting of sample chads from a sliced sample;
a sample corer comprising an elongated hollow probe with a taper opening; and
a three-axis manipulator fixably coupled to the sample corer, the three-axis manipulator being configured to move along (i) a first plane parallel to a sliced sample of 100-micron to 500-micron thickness and (ii) a second plane that bisects the sliced sample, wherein the sliced sample is placed on a cutting substrate comprising a gelling agent substrate and in a cutting solution.

17. The system of claim 16, wherein the three-axis manipulator is motorized.

18. The system of claim 16, wherein the three-axis manipulator is manually actuated.

19. The system of claim 16, wherein the sliced 100-micron to 500-micron thick section is immersed in the cutting solution comprising artificial cerebrospinal fluid (aCSF) when placed on the cutting substrate.

20. The system of claim 19, wherein the brain chad is subsequently subjected to protein extraction, immuno (Western) blotting, or a BCA assay.
